(19) United States
(12) Patent Application Publication
(10) Pub. No.: US 2015/0275147 A1
(43) Pub. Date: Oct. 1, 2015

(54) Method for Manual Dish Wash

(71) Applicant: NOVOZYMES A/S, Bagsvaerd (DK)

(72) Inventor: Nickolass Bitsch Schack, Copenhagen (DK)

(21) Appl. No.: 14/433,872

(22) PCT Filed: Oct. 25, 2013
(86) PCT No.: PCT/EP2013/072395

§ 371 Date: Apr. 7, 2015

(30) Foreign Application Priority Data
Oct. 25, 2012 (EP) 12189975.1

(51) Int. Cl. C11D 3/386; C12N 9/26

(52) U.S. Cl. CPC C11D 3/386; C12N 9/2414

(57) ABSTRACT

Disclosed is an improved method for manual dish wash where the soiled dishware is soaked in a detergent solution comprising one or more enzymes followed by brushing. The method of the invention may lead to an improved cleaning of the dishware or alternatively can the same cleaning benefit be achieved using less brushing activity compared with a similar dish washing procedure performed without one or more enzymes included in the detergent solution.

### FIELD OF THE INVENTION

The present invention relates to dish wash in particular manual dishwash (MDW).

### BACKGROUND OF THE INVENTION

Dish washing is a well know activity that takes place in practically all places where food is prepared or consumed. Traditionally dish washing is done manually by immersing the soiled dishware in dishwater, brushing or other mechanical action to the dishware followed by rinsing and drying the cleaned dishware.

Despite that automated dishwashers have been available for several years, many people around the world are still doing dishwashing manually. Especially pots and pans and other kitchenware used to heat food either on the stove or in the oven are often considered annoying to clean, because they have to soak for a long time and require a high mechanical effort to be cleaned.

There is therefore a need for improved method for dish wash in order to ease the dish washing task and to reduce the amount of mechanical action needed to obtain a satisfactory result when cleaning the dishes.

### SUMMARY OF THE INVENTION

The invention provides an improved method for manual dish wash comprising the steps of:
a. Providing a solution of a detergent composition for manual dish wash comprising one or more enzymes;
b. Soaking the soiled dishware into the solution for a period between 30 seconds and 120 minutes, preferably between 1 minute and 60 minutes preferably between 1 minute and 30 minutes, most preferred between 1 minute and 10 minutes;
c. Applying mechanical action in order to release the stains; and
d. Rinsing and drying the clean dishware.

It has surprisingly been found that using the method of the invention that a considerable improved cleaning of the dishware can be achieved compared with dish washing under the same conditions but with the omission of the one or more enzymes in the detergent composition, or that the same cleaning effect can be achieved with application of significant less mechanical action compared with dishwashing under the same conditions but without one or more enzymes in the detergent composition.

Furthermore, the invention provides a detergent composition for manual dish wash comprising at least one surfactant and one or more enzymes selected among protease, lipase, cutinase, amylase, carbohydrase, cellulase, pectinase, mannanase, arabinase, galactanase, xylanase, oxidase, e.g., laccase, and/or peroxidase.

### DETAILED DESCRIPTION OF THE INVENTION

### Definitions

Dish washing composition: The term “dish washing composition” refers to all forms of compositions for cleaning hard surfaces in particular dish ware. The present invention is not restricted to any particular type of dish wash composition or any particular detergent.

Detergent composition: The term “detergent composition” refers to all form of detergent composition for manually cleaning dish ware. The detergent composition may be in solid or in liquid form, including gels. In use the detergent composition is dissolved is an amount of water to form the ready to use wash solution.

Dish wash: The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish ware: The term dish ware is intended to mean any form of kitchen utensil, dinner set or tableware such as but not limited to pans, plates, cops, knives, forks, spoons, porcelain etc.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

### DESCRIPTION OF THE INVENTION

The method for manual dish wash according to the invention comprises the steps of:
a. Providing an solution of a detergent composition for manual dish wash comprising one or more enzymes;
b. Soaking the soiled dishware in to solution for a period between 30 seconds and 120 minutes, preferably between 1 minute and 60 minutes preferably between 1 minute and 30 minutes, most preferred between 1 minute and 10 minutes; and
c. Applying mechanical action in order to release the stains; and
d. Rinsing and drying the dishware.

Manual dish wash may be performed in several different ways and the invention is not limited to any particular setting of the dish was. In general manual dish wash is performed by immersing or contacting the soiled dishware with dishwater, using some kind of mechanical action to release the stains from the dishware, for example using a brush or sponge, followed by rinsing and drying the dishware. Manual dish wash is generally performed in some kind of open container such as a sink, a tub or a bowl, in contrast to automated dish wash that takes place inside of an automated dishwasher machine.

In step a. of the method of the invention a solution of a detergent composition for manual dish wash comprising one or more enzymes is provided.

The invention is not limited to any particular composition of the detergent composition for manual dish wash and any such composition as known in the art may be used according to the invention. In case that the detergent composition for manual dish wash does not comprise enzymes the solution may be provided by mixing the detergent composition with water and a further composition comprising one or more enzymes. However, it is preferred that the detergent composition is a detergent composition comprising one or more enzymes.

The skilled person will appreciate that a detergent composition may be a highly concentrated composition or it may be a less concentrated composition and it is therefore appreciated that the amount of the detergent composition added to the solution will depend on the particular detergent composition.

Thus in general the detergent composition is added in an amount of between 0.1 g and 2 g per liter water, such as between 0.2 g and 1.5 g per liter water, such as between 0.3 g and 1.5 g pr liter water. Highly concentrated detergent compositions for manual dish wash is typically dosed in amount of 0.3-0.5 g/l and less concentrated detergent compositions are typically dosed at 0.7 to 1.5 g/l.

The water supply for the dish wash is in general the local tap water having the composition characteristic for each geographical location. Thus, the invention is not restricted by any particular composition of the water source, but preferably the detergent composition and the amount of detergent composition is adapted to the local water quality. It is within the skills of the average detergent producers to design a detergent composition for manual dish wash that is suitable for a particular local water quality, and in general the instructions of the manufacturer of the detergent compositions can be followed in order to find a suitable amount of detergent composition.

The one or more enzymes may in principle be any enzyme that is capable of degrading or modifying a component of soils originating from food sources. Preferably the one or more enzymes are selected among: protease, lipase, acyltransferase, cutinase, an amylase, carbohydrase, cellulase, endoglucanase, xyloglucanase, pectinase, mannanase, arabinase, galactanase, xanthanase, xanthan lyase xylanase, oxidase, e.g., a laccase, and/or peroxidase. Preferably the one or more enzymes comprises at least one protease or at least one amylase and even more preferred at least one protease and one amylase.

A particular preferred embodiment uses the method of the invention a detergent composition comprising a protease and an alpha-amylase. It has surprisingly been discovered that proteases and alpha-amylases have a synergistic effect in the method of the invention on at least some stains, i.e. the effect of the combination of the two enzymes is larger than the sum of the effects of the individual enzymes.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Proteases: Suitable proteases include those of animal, vegetable or microbial origin. Microbial origin is preferred. Chemically modified or protein engineered mutants are included. The protease may be a serine protease or a metalloprotease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase™, Savinase™ Primase™, Duralase™, Esperase™, and Kannase™, Everlase (Novozymes NS), Maxatase™ Maxacal™, Maxapem™, Properase™, Purafect™, Purafect OxP™, FN2™, and FN3™ (Genencor International Inc.).

Amylases: Suitable amylases (α and/or β) include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Other useful amylases are the alpha-amylase having SEQ ID NO: 1 (corresponding to SEQ ID NO: 12 in WO01/66712) or a variant having at least 80% sequence identity to SEQ ID NO: 1 and having a substitution, a deletion or an insertion of one amino acids downstream for the amino acid corresponding to the positions in the amylase having SEQ ID NO: 1: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include such an variant having a deletion of D183 and G184 and having the substitutions R118K, N195F, R320K and R458K and a variant additionally having substitutions in one or more position selected from the group: M9, G149, G182, G186, M202, T257, Y295, N299, M323, E345 and A339, most preferred a variant that additionally having substitutions in all these positions. (Stainzyme)

Commercially available amylases are Stainzyme™, Stainzyme™ Plus, Natalase™′ Duramyl™, Termamyl™, Fungamyl™ and BAN™ (Novozymes NS), Rapidase™, Purastar™ and Purastar OxAm (from Genencor International Inc.).

Lipases and Cutinases: Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g. H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO2007/087508 and WO 2009/109500.

Preferred commercially available lipase enzymes include Lipolase™, Lipolase Ultra™′ and Lipex™; Lecitase™, Lipolex™; Lipoclean™, Lipoprime™ (Novozymes NS). Other commercially available lipases include Lumafast (Genencor Int Inc); Lipomax (Gist-Brocades/Genencor Int Inc) and Bacillus sp lipase from Solvay.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include Celluzyme™, and Carezyme™ (Novozymes NS), Clazinase™, and Puradax HA™ (Genencor International Inc.), KAC-500(B)™ (Kao Corporation) and BioTouch™ (AB Enzymes).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes NS).

The one or more enzymes acts in the method of the invention to degrade or modify the stains of the dishware so that the stains are easier released or dissolved into the dishwater.

The one or more enzymes are added in sufficient amounts to obtain a satisfactory degradation or modification in a reasonable short time. It will be appreciated that using high enzyme concentration will lead to a short holding time in step b., whereas with a lower enzyme concentration a longer holding time is needed in step b. in order to obtain same degradation of the stains.

In general each of the one or more enzymes are added in an amount corresponding to 0.001-100 mg of protein, such as 0.01-100 mg of protein, preferably 0.005-50 mg of protein, more preferably 0.01-25 mg of protein, even more preferably 0.05-10 mg of protein, most preferably 0.05-5 mg of protein, and even most preferably 0.01-2 mg of protein per liter of dishwash solution.

In one embodiment, the composition comprises enzymes in combination with one or more additional manual dishwashing composition components. The choice of additional components is within the skill of the artisan and includes conventional ingredients, including the exemplary non-limiting components set forth below.

The non-limiting list of composition components illustrated hereinafter are suitable for use in the compositions and methods herein may be desirably incorporated in certain embodiments of the invention, e.g. to assist or enhance cleaning performance, for treatment of the substrate to be cleaned, or to modify the aesthetics of the composition as is the case with perfumes, colorants, dyes or the like. The levels of any such components incorporated in any compositions are in addition to any materials previously recited for incorporation. The precise nature of these additional components, and levels of incorporation thereof, will depend on the physical form of the composition and the nature of the cleaning operation for which it is to be used. Although components mentioned below are categorized by general header according to a particular functionality, this is not to be construed as a limitation, as a component may comprise additional functionalities as will be appreciated by the skilled artisan.

Unless otherwise indicated the amounts in percentage is by weight of the composition (wt %). Suitable component materials include, but are not limited to, surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, hueing dyes, perfumes, perfume delivery systems, structure elasticizing agents, fabric softeners, carriers, hydrotropes, processing aids, solvents and/or pigments. In addition to the disclosure below, suitable examples of such other components and levels of use are found in U.S. Pat. No. 5,576,282, U.S. Pat. No. 6,306,812, and U.S. Pat. No. 6,326,348 hereby incorporated by reference.

Thus, in certain embodiments the invention do not contain one or more of the following adjuncts materials: surfactants, soaps, builders, chelating agents, dye transfer inhibiting agents, dispersants, additional enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, perfume delivery systems, structure elasticizing agents, fabric softeners, carriers, hydrotropes, processing aids, solvents and/or pigments. However, when one or more components are present, such one or more components may be present as detailed below:

In one embodiment of the invention the detergent composition comprises one or more components selected among: polymers, pH regulating agent, perfume and color.

The temperature of the solution of a detergent composition for manual dish wash comprising one or more enzymes is in general selected so the user burns his or hers hands. On the other side the temperature is preferably selected sufficiently high to ease release of in particular lipids from the dishware. Thus in general the temperature may be in the range of 0° C. and 90° C., however it is preferred that the temperature is selected in the range of 10° C. to 65° C., such as 15° C. to 60° C., such as 15° C. to 55° C., such as 15° C. to 45° C.

In step b. the dishware is soaked in the solution of a detergent composition for manual dish wash comprising one or more enzymes until the stains are degraded in a sufficient degree to dissolve in the solution or at least to be easily releasable from the dishware by mechanical action of e.g. a brush or a sponge. It will be appreciated that the soaking period depends on the concentration of the one or more enzymes in the solution, however, in general the soaking time is in the range between 30 seconds and 120 minutes, preferably between 1 minute and 60 minutes preferably between 1 minute and 30 minutes, most preferred between 1 minute and 10 minutes.

The soaking may be performed by immersing the soiled dishware in the solution of a detergent composition for manual dish wash comprising one or more enzymes, by wetting the dishware with solution of a detergent composition for manual dish wash comprising one or more enzymes e.g. by spraying the solution on the dishware; by dipping the dishware in solution of a detergent composition for manual dish wash comprising one or more enzymes followed by leaving the dishware for the period, or by other methods where the dishware is in contact with solution of a detergent composition for manual dish wash comprising one or more enzymes for the selected holding period. No particular action is required during the holding period even though it may be beneficial to agitate dishware or the solution during the period.

In step c. mechanical action is applied in order to release the stains.

The invention is not limited to any particular type of mechanical action or any particular type of tool for applying the mechanical action.

Traditionally a number of different tools have been used in manual dish wash including but not limited to a brush, a sponge or a cloth and any of these can also be applied to the present invention.

Finally in step d. the dishware is rinsed in order to remove liberated stains and dried, which may be done using methods known in the art. The rinsing may even be combined with the mechanical action applied in step c., e.g. by using a brush or a sponge for the mechanical action the user will in general dip the brush or the sponge in the dishwater before applying the mechanical action meaning that the dishwater will drain out of the brush or the sponge and rinse the soils of practically simultaneously with the mechanical action.

Drying is performed as known in the art and the invention is not limited to any particular way of drying the dishware.

The method of the invention has several benefits compared with prior art methods for manual dish wash and an improved cleaning can be achieved by the use of a certain amount of mechanical work compared to a prior art method without the use of one or more enzymes in the dishwater. Alternatively, same cleaning effect can be achieved by the use of significantly less mechanical work compared to the situation where the dish water does no comprise one or more enzymes.

### Detergent Disclosure

In one embodiment, the invention is directed to detergent compositions for use in the method of the invention, the composition comprising one or more enzymes in combination with one or more additional cleaning composition components. The choice of additional components is within the skill of the artisan and includes conventional ingredients, including the exemplary non-limiting components set forth below.

The detergent composition may comprise one or more surfactants, which may be anionic and/or cationic and/or non-ionic and/or semi-polar and/or zwitterionic and/or amphoteric, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized. When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 20% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant, typically 0.1% to 5%. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 0.1% to about 40% by weight of a zwitterionic/amphoteric surfactant typically 0.1% to 20%. Non-limiting examples of zwitterionic surfactants include betaine, alkyldimethylbetaine, sulfobetaine, and combinations thereof.

The detergent may further contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

The detergent composition may further comprise a detergent builder or co-builder, or a mixture thereof. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-10% by weight, such as about 1% to about 5%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N,N′,N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

The invention is now further described by way of examples and it should be understood that the examples are not intended to be limiting for the invention in any way.

### Examples

### Materials and Methods

### Description of the MDW Scrubbing Machine Used for Examples 1 and 2

A manual dishwash (MDW) scrubbing machine was used to test the soil removal power of different MDW detergents.

The MDW scrubbing machine (Center For Testmaterial BV, Vlaardingen, the Netherlands) consists of an electrified mechanical device onto which a normal kitchen dishwashing sponge can is mounted on a holding arm. In operation the holding arm, and hence the sponge, is moved move back and forth over a soiled tile in a reproducible uniform way for a given number of times which can be set using a counter incorporated in the scrubbing machine. The machine further comprises a slot wherein an exchangeable, flat soiled tile (approximately 10 cm*12 cm*0.5 cm) can be mounted so that it can engage with the sponge on the holding arm. At a certain position in the movement cycle of the holding arm, the sponge comes in contact with the surface of the soiled tile and is moving across the soiled tile in a reproducible way. The sponge exerts a constant pressure on the soiled tile, resembling how a person could be cleaning the surface of a given soiled piece of kitchenware during a manual dishwashing process.

Before the first cyclus of the sponge and between subsequent cycles the sponge is submerged in a solution of the MDW detergent being tested for its soil removal power.

### Description of the MDW Scrubbing Machine Used for Examples 3-11

A MDW scrubbing machine was used to test the soil removal power of different MDW detergents.

The MDW scrubbing machine used was the AB5000 abrasion and washability tester (TQC Thermimport Quality Control, Capelle aan den Ussel, the Netherlands) consists of an electrified mechanical device onto which a normal kitchen dishwashing sponge can is mounted on a holding arm. In operation the holding arm, and hence the sponge, is moved move back and forth over a soiled tile in a reproducible uniform way for a given number of times which can be set using a counter incorporated in the scrubbing machine. The machine further comprises a slot wherein an exchangeable, flat soiled tile (approximately 10 cm*12 cm*0.5 cm) can be mounted so that it can engage with the sponge on the holding arm. At a certain position in the movement cycle of the holding arm, the sponge comes in contact with the surface of the soiled tile and is moving across the soiled tile in a reproducible way. The sponge exerts a constant pressure on the soiled tile, resembling how a person could be cleaning the surface of a given soiled piece of kitchenware during a manual dishwashing process.

During the scrubbing process, there is a flow of a solution equivalent to the soil soaking solution on to the soiled tile being cleaned. The flow rate is 3 mL/min.

### Soiled Tiles

The soiled tiles used for the experiments are standard soiled melamin tiles intended for testing the cleaning power of dishwash detergents, marketed under the name of CFT Dishwash Monitors. These tiles are produced by Center For Testmaterials BV (Vlaardingen, the Netherlands). The following two tiles identified by their product number have been used for the examples:

DM 177—mix starch severe—used for examples where amylase is the sole enzyme in the detergent.

DM 06—baked diet cheese—used for examples where amylase and protease is present in the detergent.

### Description of the Method

Tiles were soaked in a detergent solution of a detergent dosed in a concentration of 0.3 g/L to 0.8 g/L, comprising the specified amount of one or more enzymes and having a starting temperature of 43° C. for a given period of time—typically 0, 10, 30, 60 or 120 minutes.

After soaking, a given tile was placed in the MDW scrubbing machine and scrubbed for a given number of times—typically 4, 8, 16 or 32 times.

After scrubbing the tile was gently rinsed under running tap water for 5 seconds and dried while lying horizontally at room temperature for at least 2 h.

After drying, the R460 value at the center of the tile was measured using a standard Color Eye apparatus (Producer: Macbeth (USA, U.K., Germany), Supplier: Largo, Model: 370).

### Enzymes Used:

Stainzyme 12 L and Savinase Ultra 16XL. Both available from Novozymes NS, Bagsvaerd, Denmark.

### Example 1

### Use of Amylase in the Method of the Invention

In order to demonstrate the benefit of alpha-amylases in manual dish wash an experiment was conducted using the method described above with the following conditions:

Detergent: Commercial Manual dishwash detergent

Initial soaking temperature (43° C.)

Water hardness: 15° dH (Ca²⁺:Mg²⁺:HCO₃⁻=4:1:7.5)

detergent concentration/dosage of 0.5 g/L

Amylase used: Stainzyme 12 L

Enzyme levels used: 0 wt %; 0.2 wt %; 0.5 wt % (dosed on top of detergent)

Number of scrubbings applied on the soil: 4 and 8

Number of repetitions for each tested combination of variables: 2

Soil removal evaluation method: Measurement of remission values at 460 nm using a standard Color Eye apparatus.

Results:

| Stainzyme 12 L | Number of | Soaking | |
|---|---|---|---|
| dosage (wt %) | scrubbings | time (min.) | R460 |
| 0.0 | 4 | 0 | 5.45 |
| | | 10 | 5.82 |
| | | 30 | 6.76 |
| | | 60 | 6.88 |
| | 8 | 0 | 11.14 |
| | | 10 | 11.56 |
| | | 30 | 16.26 |
| | | 60 | 19.20 |
| 0.2 | 4 | 0 | 3.38 |
| | | 10 | 12.11 |
| | | 30 | 25.52 |
| | 8 | 0 | 12.98 |
| | | 10 | 22.05 |
| | | 30 | 32.16 |
| 0.5 | 4 | 0 | 8.55 |
| | | 10 | 22.15 |
| | | 30 | 32.75 |
| | 8 | 0 | 12.61 |
| | | 10 | 28.88 |
| | | 30 | 36.38 |

An R460 value of 4.95 +/− 0.14 is equivalent to “no soil removal”.

A comparison of e.g. soil removal levels of “0 wt % enzyme, 60 min. soaking time, 8 scrubs” to “0.2 wt % Stainzyme 12 L, 30 min. soaking time, 4 scrubs” shows that addition of 0.2 wt % Stainzyme 12 L to the detergent solution can reduce the necessary soil soaking time and mechanical action, respectively, by at least 50% to achieve a certain level of soil removal.

A comparison of e.g. soil removal levels of “0 wt % enzyme, 60 min. soaking time, 8 scrubs” to “0.5 wt % Stainzyme 12 L, 10 min. soaking time, 4 scrubs” shows that addition of 0.5 wt % Stainzyme 12 L to the detergent solution can reduce the necessary soil soaking time by at least 80% and the necessary mechanical action by at least 50% to achieve a certain level of soil removal.

### Example 2

### Use of Amylase and Protease in the Method of the Invention

In order to demonstrate the benefit of alpha-amylases and proteases in manual dish wash an experiment was conducted using the method described above with the following conditions:

Detergent: Commercial Manual dishwash detergent

Initial soaking temperature (43° C.)

Water hardness: 15° dH (Ca²⁺:Mg²⁺:HCO₃⁻=4:1:7.5)

detergent concentration/dosage of 0.5 g/L

Amylase used: Stainzyme 12 L

Amylase levels used: 0 wt %; 0.5 wt %

Protease used: Savinase Ultra 16 XL

Protease levels used: 0 wt %; 0.5 wt %

Number of scrubbings applied on the soil: 16 and 32

Number of repetitions for each tested combination of variables: 2

Soil removal evaluation method: Measurement of remission values at 460 nm using a standard Color Eye apparatus.

Results

| | Enzyme | Number of | Soaking | |
|---|---|---|---|---|
| Enzyme | dosage (wt %) | scrubbings | time (min.) | R460 |
| None | 0.0 | 16 | 0 | 22.72 |
| | | | 30 | 23.77 |
| | | | 60 | 23.09 |
| | | | 120 | 26.70 |
| | | 32 | 0 | 19.26 |
| | | | 30 | 27.42 |
| | | | 60 | 25.56 |
| | | | 120 | 28.41 |
| Stainzyme 12 L + | 0.5 | 16 | 0 | 18.94 |
| Savinase Ultra | | | 30 | 40.93 |
| 16 XL | | | 60 | 68.71 |
| | | 32 | 0 | 29.91 |
| | | | 30 | 45.74 |
| | | | 60 | 74.79 |
| Stainzyme 12 L | 0.5 | 16 | 0 | 18.05 |
| | | | 30 | 21.25 |
| | | | 60 | 36.87 |
| | | 32 | 0 | 18.33 |
| | | | 30 | 25.88 |
| | | | 60 | 40.63 |
| Savinase Ultra | 0.5 | 16 | 0 | 20.35 |
| 16 XL | | | 30 | 20.79 |
| | | | 60 | 21.23 |
| | | 32 | 0 | 21.98 |
| | | | 30 | 21.78 |
| | | | 60 | 21.85 |

An R460 value of 11.48 +/− 0.42 is equivalent to “no soil removal”.

A comparison of e.g. soil removal levels of “0 wt % enzyme, 120 min. soaking time, 32 scrubs” to “0.5 wt % Stainzyme 12 L, 60 min. soaking time, 16 scrubs” shows that addition of 0.5 wt % Stainzyme 12 L to the detergent solution can reduce the necessary soil soaking time and mechanical action, respectively, by at least 50% to achieve a certain level of soil removal.

A comparison of e.g. soil removal levels of “0 wt % enzyme, 120 min. soaking time, 32 scrubs” to “0.5 wt % Stainzyme 12 L+0.5 wt % Savinase Ultra 16 XL, 30 min. soaking time, 16 scrubs” shows that addition of 0.5 wt % Stainzyme 12 L+0.5 wt % Savinase Ultra 16 XL to the detergent solution can reduce the necessary soil soaking time by at least 75% and the necessary mechanical action by at least 50% to achieve a certain level of soil removal.

Comparing the total soil removal level of “0.5 wt % Savinase Ultra 16 XL, 16 scrubs” and “0.5 wt % Stainzyme 12 L, 16 scrubs” to the soil removal level at “0.5 wt % Savinase Ultra 16 XL+0.5 wt % Stainzyme 12 L, 16 scrubs” after either 30 min. or 60 min. Soaking time, respectively, clearly reveals that there is a synergetic soil removal effect of protease and amylase on this soil, i.e. the soil removal level of the enzymes in combination is larger than the sum soil removal levels for the individual enzymes.

This synergy effect is also seen when comparing the soil removal levels at the same conditions but for 32 scrubs instead of 16 scrubs. The experiment demonstrates the benefits of using alpha-amylases and proteases in manual dish wash. Further, a clear synergi between amylase and protease was observed.

### Example 3

### Use of Amylase in the Method of the Invention

In order to demonstrate the benefit of alpha-amylases in manual dish wash an experiment was conducted using the method described above with the following conditions:

Detergent: Commercial manual dishwash detergent
100% detergent dosage: 0.8 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.1 wt %; 0.2 wt % (dosed on top of detergent. The enzyme levels are based on the 100% detergent dosage).
Number of scrubbings applied on the soil: 4 and 8
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage (wt %) | scrubbings | Time (min.) | R460 |
| 0.8 | 0 | 4 | 0 | 8.59 |
| | | | 10 | 10.72 |
| | | | 30 | 10.33 |
| | | | 60 | 9.96 |
| | | 8 | 0 | 9.58 |
| | | | 10 | 12.56 |
| | | | 30 | 12.40 |
| | | | 60 | 12.07 |
| 0.6 | 0.2 | 4 | 0 | 8.94 |
| | | | 10 | 14.23 |
| | | | 30 | 38.14 |
| | | | 60 | 47.96 |
| | | 8 | 0 | 9.09 |
| | | | 10 | 20.97 |
| | | | 30 | 43.23 |
| | | | 60 | 52.48 |
| 0.6 | 0.1 | 4 | 0 | 9.19 |
| | | | 10 | 11.39 |
| | | | 30 | 22.04 |
| | | | 60 | 40.79 |
| | | 8 | 0 | 9.70 |
| | | | 10 | 14.07 |
| | | | 30 | 28.62 |
| | | | 60 | 48.18 |
| 0.4 | 0.2 | 4 | 0 | 7.88 |
| | | | 10 | 13.55 |
| | | | 30 | 29.66 |
| | | | 60 | 40.38 |
| | | 8 | 0 | 7.87 |
| | | | 10 | 23.03 |
| | | | 30 | 32.58 |
| | | | 60 | 44.98 |
| 0.4 | 0.1 | 4 | 0 | 7.93 |
| | | | 10 | 9.63 |
| | | | 30 | 19.32 |
| | | | 60 | 34.02 |
| | | 8 | 0 | 8.69 |
| | | | 10 | 13.44 |
| | | | 30 | 26.31 |
| | | | 60 | 40.36 |

An R460 value of 4.95 +/− 0.14 is equivalent to “no soil removal”.

### Example 4

### Use of Amylase in the Method of the Invention

In order to demonstrate the benefit of alpha-amylases in manual dish wash an experiment was conducted using the method described above with the following conditions:

Detergent: Commercial manual dishwash detergent
Detergent dosage: 0.5 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.2 wt % (dosed on top of detergent)
Number of scrubbings applied on the soil: 4 and 8
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage (wt %) | scrubbings | Time (min) | R460 |
| 0.5 | 0.0 | 4 | 0 | 7.33 |
| | | | 10 | 11.10 |
| | | | 30 | 10.74 |
| | | | 60 | 9.88 |
| | | 8 | 0 | 9.74 |
| | | | 10 | 12.09 |
| | | | 30 | 12.00 |
| | | | 60 | 10.89 |
| 0.5 | 0.2 | 4 | 0 | 9.30 |
| | | | 10 | 12.52 |
| | | | 30 | 23.72 |
| | | | 60 | 30.02 |
| | | 8 | 0 | 11.04 |
| | | | 10 | 16.17 |
| | | | 30 | 22.75 |
| | | | 60 | 37.84 |

An R460 value of 4.95 +/− 0.14 is equivalent to “no soil removal”.

### Example 5

### Use of Amylase in the Method of the Invention

In order to demonstrate the benefit of alpha-amylases in manual dish wash an experiment was conducted using the method described above with the following conditions:

Detergent: Commercial manual dishwash detergent
100% detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.15 wt %; 0.30 wt % (dosed on top of detergent. The enzyme levels are based on the 100% detergent dosage).
Number of scrubbings applied on the soil: 4 and 8
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage wt %) | scrubbings | Time (min.) | R460 |
| 0.4 | 0.0 | 4 | 0 | 8.58 |
| | | | 10 | 9.96 |
| | | | 30 | 9.66 |
| | | | 60 | 10.28 |
| | | 8 | 0 | 8.97 |
| | | | 10 | 11.02 |
| | | | 30 | 10.71 |
| | | | 60 | 10.17 |
| 0.3 | 0.3 | 4 | 0 | 8.59 |
| | | | 10 | 11.55 |
| | | | 30 | 16.44 |
| | | | 60 | 44.51 |
| | | 8 | 0 | 9.26 |
| | | | 10 | 15.09 |
| | | | 30 | 22.34 |
| | | | 60 | 45.41 |
| 0.3 | 0.15 | 4 | 0 | 8.96 |
| | | | 10 | 11.73 |
| | | | 30 | 16.14 |
| | | | 60 | 35.63 |
| | | 8 | 0 | 10.00 |
| | | | 10 | 13.90 |
| | | | 30 | 23.30 |
| | | | 60 | 40.24 |
| 0.2 | 0.3 | 4 | 0 | 9.39 |
| | | | 10 | 12.32 |
| | | | 30 | 29.36 |
| | | | 60 | 36.49 |
| | | 8 | 0 | 9.18 |
| | | | 10 | 18.89 |
| | | | 30 | 35.33 |
| | | | 60 | 43.82 |
| 0.2 | 0.15 | 4 | 0 | 9.14 |
| | | | 10 | 10.45 |
| | | | 30 | 14.86 |
| | | | 60 | 38.03 |
| | | 8 | 0 | 8.64 |
| | | | 10 | 12.63 |
| | | | 30 | 21.42 |
| | | | 60 | 42.06 |

An R460 value of 4.95 +/− 0.14 is equivalent to “no soil removal”.

### Example 6

### Use of Amylase in the Method of the Invention

In order to demonstrate the benefit of alpha-amylases in manual dish wash an experiment was conducted using the method described above with the following conditions:

Detergent: Commercial manual dishwash detergent
100% detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.15 wt % (dosed on top of detergent. The enzyme levels are based on the 100% detergent dosage).
Number of scrubbings applied on the soil: 12 and 24
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage (wt %) | scrubbings | Time (min.) | R460 |
| 0.4 | 0.00 | 12 | 0 | 9.28 |
| | | | 10 | 9.44 |
| | | | 30 | 13.32 |
| | | | 60 | 11.55 |
| | | 24 | 0 | 11.74 |
| | | | 10 | 13.85 |
| | | | 30 | 14.08 |
| | | | 60 | 16.23 |
| 0.3 | 0.15 | 12 | 0 | 10.52 |
| | | | 10 | 14.73 |
| | | | 30 | 18.35 |
| | | | 60 | 33.80 |
| | | 24 | 0 | 11.44 |
| | | | 10 | 19.76 |
| | | | 30 | 28.30 |
| | | | 60 | 44.94 |

An R460 value of 4.95 +/− 0.14 is equivalent to “no soil removal”.

### Example 7

### Use of Amylase in the Method of the Invention

In order to demonstrate the benefit of alpha-amylases in manual dish wash an experiment was conducted using the method described above with the following conditions:

Detergent: Commercial manual dishwash detergent
100% detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.30 wt % (dosed on top of detergent. The enzyme levels are based on the 100% detergent dosage).
Number of scrubbings applied on the soil: 12 and 24
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage (wt %) | scrubbings | Time (min.) | R460 |
| 0.4 | 0.00 | 12 | 0 | 10.03 |
| | | | 10 | 10.43 |
| | | | 30 | 9.59 |
| | | | 60 | 10.34 |
| | | 24 | 0 | 10.06 |
| | | | 10 | 11.20 |
| | | | 30 | 13.11 |
| | | | 60 | 12.37 |
| 0.3 | 0.30 | 12 | 0 | 8.77 |
| | | | 10 | 15.25 |
| | | | 30 | 35.73 |
| | | | 60 | 42.94 |
| | | 24 | 0 | 10.68 |
| | | | 10 | 26.91 |
| | | | 30 | 39.34 |
| | | | 60 | 50.62 |

An R460 value of 4.95 +/− 0.14 is equivalent to “no soil removal”.

### Example 8

### Use of Amylase in the Method of the Invention

In order to demonstrate the benefit of alpha-amylases in manual dish wash an experiment was conducted using the method described above with the following conditions:

Detergent: Commercial manual dishwash detergent
100% detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Enzyme levels used: 0 wt %; 0.30 wt % (dosed on top of detergent. The enzyme levels are based on the 100% detergent dosage).
Number of scrubbings applied on the soil: 12 and 24
Number of repetitions for each tested combination of variables: 2
Soil removal evaluation method: Measurement of remission values at 460 nm using a standard Color Eye apparatus.

### Results:

| Detergent | Stainzyme 12 L | Number of | Soaking | |
|---|---|---|---|---|
| dosage (g/L) | dosage (wt %) | scrubbings | Time (min.) | R460 |
| 0.4 | 0 g | 24 | 0 | 8.72 |
| | | | 10 | 10.38 |
| | | | 30 | 10.66 |
| | | | 60 | 9.28 |
| 0.3 | 0.3 | 12 | 0 | 10.01 |
| | | | 10 | 12.09 |
| | | | 30 | 12.51 |
| | | | 60 | 38.80 |
| 0.3 | 0.3 | 24 | 0 | 12.00 |
| | | | 10 | 22.27 |
| | | | 30 | 22.88 |
| | | | 60 | 44.74 |

An R460 value of 4.95 +/− 0.14 is equivalent to “no soil removal”.

### Example 9

### Use of Amylase and Protease in the Method of the Invention

In order to demonstrate the benefit of alpha-amylases and proteases in manual dish wash an experiment was conducted using the method described above with the following conditions:

Detergent: Commercial manual dishwash detergent
100% detergent dosage: 0.8 g/L
Initial soaking temperature (43° C.)
Water hardness: 15° dH (Ca²⁺:Mg²⁺:HCO₃⁻=4:1:7.5)
Amylase used: Stainzyme 12 L
Amylase levels used: 0 wt %; 0.20 wt % (dosed on top of detergent. The enzyme levels are based on the 100% detergent dosage).
Protease used: Savinase Ultra 16 XL
Protease levels used: 0 wt %; 0.20 wt % (dosed on top of detergent. The enzyme levels are based on the 100% detergent dosage).
Number of scrubbings applied on the soil: 32
Number of repetitions for each tested combination of variables: 2
Soil removal evaluation method: Measurement of remission values at 460 nm using a standard Color Eye apparatus.

| Detergent | | Enzyme | Number | Soaking | |
|---|---|---|---|---|---|
| dosage | | dosage dosage | of | Time | |
| (g/L) | Enzyme | (wt %) | scrubbings | (min.) | R460 |
| 0.8 | None | 0 | 32 | 0 | 15.39 |
| | | | 32 | 60 | 18.22 |
| | | | 32 | 120 | 19.81 |
| 0.6 | Stainzyme | 0.2 | 32 | 0 | 15.14 |
| | 12L | | 32 | 60 | 20.43 |
| | | | 32 | 120 | 46.15 |
| 0.6 | Savinase | 0.2 | 32 | 0 | 14.95 |
| | 16 Ultra XL | | 32 | 60 | 21.60 |
| | | | 32 | 120 | 19.46 |
| 0.6 | Stainzyme | 0.2 and 0.2 | 32 | 0 | 15.25 |
| | 12 L and | | 32 | 60 | 30.71 |
| | Savinase | | 32 | 120 | 61.94 |
| | Ultra 16 XL | | | | |

An R460 value of 11.48 +/− 0.42 is equivalent to “no soil removal”.

### Example 10

### Use of Amylase and Protease in the Method of the Invention

In order to demonstrate the benefit of alpha-amylases and proteases in manual dish wash an experiment was conducted using the method described above with the following conditions:

Detergent: Commercial manual dishwash detergent
100% detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Amylase levels used: 0 wt %; 0.30 wt % (dosed on top of detergent. The enzyme levels are based on the 100% detergent dosage).
Protease used: Savinase Ultra 16 XL
Protease levels used: 0 wt %; 0.30 wt % (dosed on top of detergent. The enzyme levels are based on the 100% detergent dosage).
Number of scrubbings applied on the soil: 32
Number of repetitions for each tested combination of variables: 2
Soil removal evaluation method: Measurement of remission values at 460 nm using a standard Color Eye apparatus.

### Results:

| Detergent | | Enzyme | Number | Soaking | |
|---|---|---|---|---|---|
| dosage | | dosage | of | Time | |
| (g/L) | Enzyme | (wt %) | scrubbings | (min.) | R460 |
| 0.4 | None | 0 | 32 | 0 | 15.74 |
| | | | 32 | 60 | 18.95 |
| | | | 32 | 120 | 18.54 |
| 0.3 | Stainzyme | 0.3 | 32 | 0 | 15.27 |
| | 12L | | 32 | 60 | 20.30 |
| | | | 32 | 120 | 31.29 |
| 0.3 | Savinase | 0.3 | 32 | 0 | 13.34 |
| | 16 Ultra XL | | 32 | 60 | 16.78 |
| | | | 32 | 120 | 18.67 |
| 0.3 | Stainzyme | 0.3 and 0.3 | 32 | 0 | 13.63 |
| | 12 L and | | 32 | 60 | 18.28 |
| | Savinase | | 32 | 120 | 44.43 |
| | Ultra 16 XL | | | | |

An R460 value of 11.48 +/− 0.42 is equivalent to “no soil removal”.

### Example 11

### Use of Amylase and Protease in the Method of the Invention

In order to demonstrate the benefit of alpha-amylases and proteases in manual dish wash an experiment was conducted using the method described above with the following conditions:

Soil type: Knorr Quattro Formaggi®—4 Sorten Káse & Basilikum (ingredients: Vegetable fat, flour (wheat, corn), 19% cheese mix (blue cheese, mozzarella, hard cheese, Crescenza cheese, whey product, cheese), starch, iodized table salt, lactose, milk protein, yeast extract, table salt, onions, sugar, herbs, white wine extract, spices, aroma, glucose)
Soil preparation method: The content of 1 sauce bag is mixed with 250 mL water using a whisk. While stirring, the mixture is heated in a pot on a stove and allowed to boil for one minute. After cooling, the sauce is applied onto clean stainless steel tiles using a paint roller to create an even layer of the sauce on the tiles. The soiled tiles are heated in an oven at 150° C. for 30 minutes. After cooling to room temperature the tiles are ready for use.
Detergent: Commercial manual dishwash detergent
Detergent dosage: 0.4 g/L
Initial soaking temperature (43° C.)
Water hardness: 15° dH (Ca²⁺:Mg²⁺:HCO₃⁻=4:1:7.5)
Amylase used: Stainzyme 12 L
Amylase levels used: 0 wt %; 0.30 wt % (dosed on top of detergent)
Protease used: Savinase Ultra 16 XL
Protease levels used: 0 wt %; 0.30 wt % (dosed on top of detergent)
Number of scrubbings applied on the soil: 32
Number of repetitions for each tested combination of variables: 2
Soil removal evaluation method: Visual Scoring Units (0=no soil removal, 10=complete soil removal).

### Results:

| Detergent | | Enzyme | | Soaking | |
|---|---|---|---|---|---|
| dosage | | dosage | Number of | time | Visual |
| (g/L) | Enzyme | (wt %) | scrubbings | (min.) | Scoring |
| 0.4 | None | 0 | 32 | 60 | 0 |
| | | | 32 | 90 | 1 |
| | | | 32 | 120 | 2 |
| 0.4 | Stainzyme | 0.3 | 32 | 60 | 3 |
| | 12L | | 32 | 90 | 4 |
| | | | 32 | 120 | 5 |
| 0.4 | Savinase | 0.3 | 32 | 60 | 2 |
| | Ultra 16 XL | | 32 | 90 | 2 |
| | | | 32 | 120 | 2 |
| 0.4 | Stainzyme | 0.3 and 0.3 | 32 | 60 | 6 |
| | 12 L and | | 32 | 90 | 7 |
| | Savinase | | 32 | 120 | 8 |
| | Ultra 16 XL | | | | |

1-14. (canceled)

15. A method for manual dish wash comprising the steps of:
(a) providing a solution of a detergent composition for manual dish wash comprising one or more enzymes;
(b) soaking the soiled dishware into the solution for a period between 30 seconds and 120 minutes, preferably between 1 minute and 60 minutes preferably between 1 minute and 30 minutes, most preferred between 1 minute and 10 minutes; and
(c) applying mechanical action in order to release the stains; and
(d) rinsing and drying.

16. The method of claim 15, wherein the one or more enzymes are selected from the group consisting of amylase, arabinase, carbohydrase, cellulase, cutinase, galactanase, lipase, mannanase, oxidase (e.g., laccase, and/or peroxidase), pectinase, protease, and xylanase.

17. The method of claim 16, wherein the one or more enzymes are selected from the group consisting of amylase and protease.

18. The method of claim 17, wherein the one or more enzymes consists of a protease and/or an amylase.

19. The method of claim 18, wherein the amylase is a variant having at least 80% sequence identity to SEQ ID NO: 1 and having a substitution, a deletion or an insertion of one amino acids downstream for the amino acid corresponding to the positions in the amylase having SEQ ID NO: 1: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484.

20. The method of claim 19, wherein the amylase is a variant having at least 80% sequence identity to SEQ ID NO: 1 having a deletion of D183 and G184 and having the substitutions R118K, N195F, R320K and R458K or a variant additionally having substitutions in one or more positions selected from the group: M9, G149, G182, G186, M202, T257, Y295, N299, M323 and A339.

21. The method of claim 15, wherein each of the one or more enzymes is added in an amount corresponding to 0.001-100 mg of protein per liter of the solution.

22. The method of claim 15, wherein the mechanical action is applied using a brush, a sponge or a cloth.

23. The method of claim 15, wherein the initial temperature of the solution in step (a) is in the range of 0° C. and 90° C.

24. A detergent composition for manual dish wash comprising at least one surfactant and one or more enzymes selected from the group consisting of amylase, arabinase, carbohydrase, cellulase, cutinase, galactanase, lipase, mannanase, oxidase (e.g., laccase, and/or peroxidase), pectinase, protease, and xylanase.

25. The detergent composition of claim 24, comprising at least one protease and at least one amylase, preferably an alpha-amylase.

26. The detergent composition of claim 25, wherein the amylase is a variant having at least 80% sequence identity to SEQ ID NO: 1 and having a substitution, a deletion or an insertion of one amino acids downstream for the amino acid corresponding to the positions in the amylase having SEQ ID NO: 1: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484.

27. The composition of claim 24, wherein the amylase is a variant having at least 80% sequence identity to SEQ ID NO: 1 having a deletion of D183 and G184 and having the substitutions R118K, N195F, R320K and R458K and a variant additionally having substitutions in one or more positions selected from the group: M9, G149, G182, G186, M202, T257, Y295, N299, M323 and A339, most preferred a variant that additionally having substitutions in all these positions.

28. The detergent composition of claim 24, further comprising one or more components selected among: polymers, pH regulating agent, perfume and color.


SEQUENCE LISTING (1 sequence)

SEQ ID NO: 1 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK